(19) United States
(12) Patent Application Publication
(10) Pub. No.: US 2006/0006033 A1
(43) Pub. Date: Jan. 12, 2006

(54) Images affixed to luggage wheels easy access to closed luggage

(76) Inventor: Anthony Gerome Edwards, Marina Del Rey, CA (US)

Correspondence Address: ANTHONY EDWARDS, 13700 TAHITI WAY #153, MARINA DEL REY, CA, 90292, US

(21) Appl. No.: 10/791,124

(22) Filed: Jul. 8, 2004

(51) Int. Cl. A61K 31/4965

(52) U.S. Cl. 190/18 A

(57) ABSTRACT

The affixing of an image such as a photo, flag or school colors to the outer surface of luggage wheels. The providing of easy access to closed luggage through a zippered opening through the upper end of the cover or lid of said closed luggage.

A flat material of suitable thickness and in the appropriate size and shape of the outer surface of the wheel is provided with adhesive material on both sides that is protected by a removable covering. The covering on one side is removed exposing the adhesive material and the image that is to be affixed to it is applied. The protective covering is then removed from the other side exposing the adhesive material and the image is applied to the outer surface of the wheel.

Easy access is provided to the interior of a closed luggage case by installing a zipper which runs for any desired length up one side of the front of the luggage case, across the upper end of the luggage case and down the opposite side of the luggage case for a distance equal to the length of the zipper on the opposite side of the luggage case providing easy access to the interior of the luggage case whether it is lying in a horizontal position or standing upright in a vertical position. The small pocket that is usually affixed to the front upper end of a conventional luggage case may still be applied to the flap that is created by the introduction of this zipper.

### BRIEF DESCRIPTION OF THE DRAWINGS

FIG. 1 is a perspective view of the embodiment of the invention showing a luggage case with a photograph affixed to the outer side of the wheel.

FIG. 2 is a front elevational view of a luggage case with a conventional pocket affixed to the outer side of the upper half of the cover of the luggage case for storage of documents etc.

### DESCRIPTION OF PREFERRED EMBODIMENT

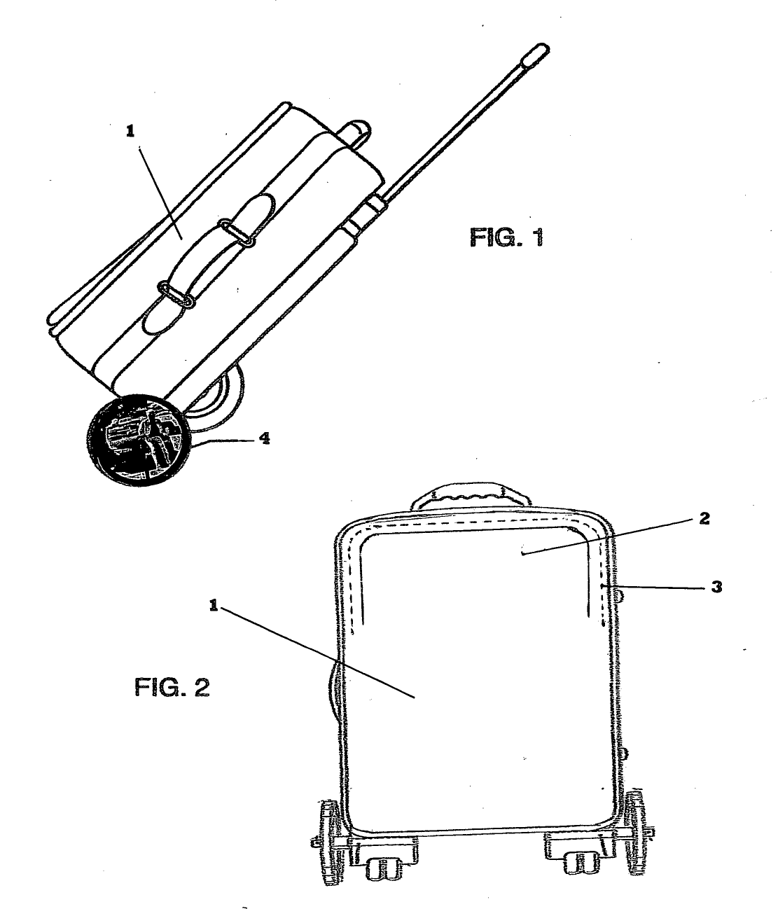

As shown in the drawings FIG. 1, a preferred embodiment of the invention, has a side view of a luggage case 1 with photo image affixed to the outer surface of wheel 4.

As shown in the drawings FIG. 2, a preferred embodiment of the invention shows a front view of luggage case 1 with a conventional small pocket 2 in the upper half of luggage case 1 and a broken line 3 defining the zippered access in the front or cover of closed luggage case 1.

I claim:

1. In a conventional soft luggage case, the improvement comprising:
1. the affixing of an image such as a photo, flag or school colors to the outer surface of the wheels of said luggage.
2. easy access to a closed luggage case through a zippered opening in the upper end of the front or cover of said luggage case.
